(19) United States
(12) Patent Application Publication
(10) Pub. No.: US 2016/0346018 A1
(43) Pub. Date: Dec. 1, 2016

(54) DEFECT FIXATION DEVICE

(71) Applicant: The Texas A &M University System, College Station, TX (US)

(72) Inventors: Carl A. Gregory, Belton, TX (US); Bret H. Clough, Temple, TX (US); Roland Kaunas, College Station, TX (US); Robert Reese, Bryan, TX (US); Abhishek Tondon, Houston, TX (US); Christopher Dennis Chaput, Temple, TX (US)

(73) Assignee: The Texas A &M University System, College Station, TX (US)

(21) Appl. No.: 15/170,926

(22) Filed: Jun. 1, 2016

Related U.S. Application Data
(60) Provisional application No. 62/169,110, filed on Jun. 1, 2015.

(51) Int. Cl. A61B 17/72

(52) U.S. Cl. CPC A61B 17/7233

(57) ABSTRACT

An intermedullary device and methods of use are described. The device is configured for fixation of a long bone and to facilitate healing of the long bone. The device comprises an elongate member having a first end, a second end, and a mid region. The mid region comprises a band or collar extending outwardly away from the outer surface of the device to increase all or a portion of the cross-sectional diameter of the mid region. The elongate member has a length that may be about the same as or less than the length of the long bone's diaphyseal region. The band further comprises at least one engaging surface configured for engaging with a cortical region of the long bone at a defect site. Use of the device facilitates healing of the long bone at or near the defect site. The method comprises providing a first end of the device in a first exposed region of the long bone's medullary canal. The method further comprises providing a second end of the device in a second exposed region of the medullary canal.

### CROSS-REFERENCE TO RELATED APPLICATION

This application claims the benefit of priority to U.S. Provisional Application No. 62/169,110, the entirety of which is incorporated herein by reference to the maximum extent.

### STATEMENT REGARDING FEDERALLY SPONSORED RESEARCH OR DEVELOPMENT

The disclosed was made in part with government support under R01AR066033-01 awarded by the National Institutes of Health. The government has certain rights in the invention.

### TECHNICAL FIELD

Disclosed herein are fixation and stabilization devices for insertion in a medullary space of a long bone, such as the femur.

### BACKGROUND

Long bone fractures are expected to increase in an ever growing population, with an increasing aging population and a population more active and involved in sports and dynamic or vigorous activities. Unfortunately, up to 30% of all long bone fractures fail, resulting in non-union of the bone, a serious, detrimental and costly consequence. To better understand serious bone injuries, mammalian models are employed in biomedical research. Many mammal types are used. Due to their small size, establishment of stabilized bone defects or lesions in smaller mammals, such as mice are beyond the capabilities of most research groups. There remains a need for defect stabilization devices and methods that may be used in both small and large mammals. Said devices should allow for mimicking of serious bone trauma in the long bone, and allow for endochondral ossification during the healing process. Said devices should not be challenging to develop, or to install for use.

### SUMMARY

Describe herein is a fixation and stabilization device that meets the needs described above. The devices described herein allow for mimicking of serious bone trauma in the long bone of a mammal and allow for endochondral ossification during the healing process. The devices described herein minimize damage to the distal ends of the long bone. Said devices do not require extra fixation (external fixation or any additional screws, locking plates, etc.).

Processes for developing the devices with ease are described. Said processes provide precise sizings, for improved fitting in the medullary space of the long bone. Said processes allow for optimization of the devices for strain (type of mammal), gender and age. With the sizings and fittings described herein, the devices described herein minimize torsional (rotational) and/or axial motion, which is found in many of the alternative intermedullary devices.

The described devices are easier to install and provide a means for improved analysis of the defect after installation. The devices described herein include one or a number of landmarks that define the origins of an initial defect, allowing for accurate analysis and continued accuracy during healing.

In one or more forms is an intermedullary device configured for fixation of a long bone comprising an elongate member having a first end, a second end, and a mid region. The mid region compris a band extending outwardly from the elongate member and increasing a cross-sectional diameter about at least a portion of the mid region. The band may comprise an engaging surface configured for engaging with a cortical region of the long bone. The elongate member may have a length from the first end to the second end that is about a length of the long bone's diaphyseal region. The device in use is so configured that it may not penetrate a proximal or distal epiphysis region of the long bone. The device in use is so configured that it does not damage a metaphyseal region of the long bone. The band has a length that may be from about 25% to about 50% of a length of the intermedullary device. The band may comprise more than one band. The device is so configured that it may not require fixation using an external fixation element. The device is so configured that it may not require bores or holes therein for mating with an anchoring element. The band may be comprised of a same material as the elongate member. The band may be comprised of a different material than that of the elongate member. The elongate member, excluding the band, may have on average a cross-sectional diameter that is about or similar to a cross-sectional diameter of the long bone's medullary canal. The device may further comprise one or more surface features, including one or more of the group selected from texturing, roughening, grooves, tabs, and barbs. The device may further comprise one or more surface coatings applied on at least a portion of the intermedullary device.

Additionally, described herein is a device configured for positioning in a medullary canal of a long bone to divide the long bone into a proximal section having an exposed cortical region and a distal section having an exposed cortical region, with a spaced apart region separating the proximal section and the distal section. The device has a cross-sectional diameter at a mid region that is at least 10% greater than any other cross-sectional diameter of the device and is so configured to engage with one or more of at least a portion of the exposed cortical region of the proximal section of the long bone and at least a portion of the exposed cortical region of the distal section of the long bone. The length of the device may be configured to extend from a proximal end of the long bone's diaphysis to a distal end of the long bone's diaphysis. The mid region of the device may engage with the exposed cortical region the proximal section by abutting at least a portion of the exposed cortical region. The mid region of the device may engage with the exposed cortical region the distal section by abutting at least a portion of the exposed cortical region. The spaced apart region may have a length that is from about 25% to about 50% the length of the device and the mid portion of the device spans the length of the spaced apart region.

In further embodiments is described a method of facilitating healing of a long bone using an intermedullary device. The method comprises providing a first end of the intermedullary device in a first exposed region of a medullary canal of the long bone, the first exposed region formed at a mid portion of the long bone, the intermedullary device having a mid region with a cross-sectional diameter that is at least 10% greater than any other cross-sectional diameter of the intermedullary device. The method further comprises providing a second end of the intermedullary device in a second exposed region of the medullary canal of the long bone, the second exposed region formed at a mid portion of the long bone. The method may further comprise causing cortical bone about the first exposed region to be proximate to a first engaging surface of the mid region of the intermedullary device. The method may further comprise causing cortical bone about the second exposed region to be proximate to a second engaging surface of the mid region of the intermedullary device. In the method, the providing of the first end of the intermedullary device may extend a proximal end of the intermedullary device so it is at or proximate to a proximal end of the long bone's diaphysis and providing the second end of the intermedullary device extends a distal end of the intermedullary device so it is at or proximate to a distal end of the long bone's diaphysis. In the method, the causing of the cortical bone about the first exposed region may include abutting the first engaging surface of the mid region of the intermedullary device with at least portion of the cortical bone about the first exposed region. In the method, the causing cortical bone about the second exposed region may include abutting the second engaging surface of the mid region of the intermedullary device with at least portion of the cortical bone about the second exposed region. The method may further comprise locking the intermedullary device at the mid region.

In one or more embodiments is an intermedullary device configured for fixation of a bone, the device comprising an elongate member having a first end, a second end, and a mid region. The mid region may comprise a band extending outwardly from the elongate member, and increasing a cross-sectional diameter about at least a portion of the mid region. The band may comprise an engaging surface configured for engaging with a cortical region of the bone. The elongate member may have a length from the first end to the second end that is about or near about a length of a diaphyseal region of the bone. The device in use may be so configured that it does not penetrate one or more of a proximal epiphyseal region of the bone or distal epiphyseal region of the bone. The device in use may be so configured that it does not damage a metaphyseal region of the bone. The band may have a length that is from about 15% to about 50% of a length of the intermedullary device. The band may comprise more than one band. The intermedullary device may be configured for use without an external fixation element. The intermedullary device is configured for use without any external fixation element. The intermedullary device may be configured for use without anchoring bores therein, or without a separate anchoring element. The intermedullary device is configured for use without any anchoring bores therein, or without any separate anchoring element. The band may be comprised of a same material as the elongate member. The band may be comprised of a different material than the elongate member. The elongate member, excluding the band, may have on average a cross-sectional diameter that is about or similar to an average cross-sectional diameter of a medullary canal of the bone. The intermedullary device may further comprise one or more surface features, including one or more of the group comprising a texturing, a roughening, a groove, a slit, a tab, and a barb. The intermedullary device may further comprise at least one groove. The intermedullary device may further comprise at least two grooves. The first groove may be positioned between the first end and the mid region. The first groove may be along a length of the intermedullary device between the first end and the mid region. The length of the groove may be more than 40%, or more than 50%, or more than 60%, of the length of the intermedullary device between the first end and the mid region. The second groove may be positioned between the second end and the mid region. The second groove may be along a length of the intermedullary device between the second end and the mid region. The length of the groove may be more than 40%, or more than 50%, or more than 60%, of the length of the intermedullary device between the first end and the mid region. The intermedullary device may further comprise one or more surface coatings applied on at least a portion of the intermedullary device.

In one or more embodiments is a device configured for positioning in a medullary canal of a bone to separate the bone into a proximal section having an exposed cortical region, a distal section having an exposed cortical region, and a spaced apart region separating the proximal section of the bone from the distal section of the bone. The device comprises a first end, a second end, and a mid region, in which a cross-sectional diameter of the mid region is at least 10% greater than any other cross-sectional diameter of the device. The mid-region may be so configured to reside in the spaced apart region separating the proximal section of the bone from the distal section of the bone, and to engage with at least a portion of the exposed cortical region of the proximal section of the bone, and to further engage with at least a portion of the exposed cortical region of the distal section of the bone. The device may further comprise a length so that the first end extends to a distal end of the diaphyseal region and is proximate a distal metaphyseal region of the bone without penetrating the distal metaphyseal region, and the second end extends to a proximal end of the diaphyseal region and is proximate a proximal metaphyseal region of the bone without penetrating the proximal metaphyseal region. The device may further comprise a first groove between the first end and the mid region, and a second groove between the second end and the mid region. The mid region of the device may have a length that is from about 15% to about 50% the length of the device, and the mid portion spans a distance formed by the spaced apart region that separates the proximal section of the bone from the distal section of the bone.

In one or more embodiments is a method of configuring an intermedullary device for a bone, in which the bone comprises a proximal portion having a first medullary canal and a first exposed region, and a distal portion having a second medullary canal and a second exposed region. The method comprises configuring a first end of the intermedullary device for a first medullary canal at the first exposed region of the bone, the first exposed region being at or near a mid portion of the bone, the intermedullary device comprising a mid region and a cross-sectional diameter in the mid-region being at least 10% greater than any other cross-sectional diameter of the intermedullary device that provides a first engaging surface at a first end of the mid region of the intermedullary device and provides a second engaging surface at a second end of the mid region of the intermedullary device. The method comprises configuring a second end of the intermedullary device for a second medullary canal at the second exposed region of the bone. The method comprises configuring at least a portion of the mid region of the intermedullary device to abut at least a portion of the first exposed region after having been configured for the first medullary canal of the proximal portion of the bone in a manner for cortical bone at or near the first exposed region to be proximate to the first engaging surface of the first end of the mid region of the intermedullary device. The method configuring at least a portion mid region of the intermedullary device to be proximate to at least a portion of the second exposed region after having been configured for the second medullary canal of the distal portion of the bone in a manner for cortical bone at or near the second exposed region to be proximate to the second engaging surface of the second end of the mid region of the intermedullary device. The method comprises configuring the mid region of the intermedullary device to have a maximal cross-sectional diameter that is about or less than an average cross-sectional diameter of the mid portion of the bone. The method comprises configuring the first end and the second end of the intermedullary device to have a maximal cross-sectional diameter that is about or less than an average cross-sectional diameter of the medullary canal of the bone. The method comprises configuring the first end of the intermedullary device to have a maximal cross-sectional diameter that is about or less than an average cross-sectional diameter of a proximal portion of the medullary canal of the bone. The method comprises configuring the second end of the intermedullary device to have a maximal cross-sectional diameter that is about or less than an average cross-sectional diameter of a distal portion of the medullary canal of the bone.

In one or more embodiments is a method of configuring an intermedullary device for fixating a bone, in which the bone comprises a proximal portion having a first medullary canal and a first exposed region, and a distal portion having a second medullary canal and a second exposed region. The method comprises configuring a first end of the intermedullary device for a first medullary canal at the first exposed region, the first exposed region being at or near a mid portion of the bone, the intermedullary device comprising a mid region and a cross-sectional diameter in the mid-region being at least 10% greater than any other cross-sectional diameter of the intermedullary device that provides a first engaging surface at a first end of the mid region of the intermedullary device and provides a second engaging surface at a second end of the mid region of the intermedullary device. The method comprises configuring a second end of the intermedullary device for a second medullary canal at the second exposed region. The method comprises causing at least a portion of the mid region of the intermedullary device to be configured to abut at least a portion of the first exposed region after having been provided in the first medullary canal of the proximal portion of the bone in a manner for cortical bone at or near the first exposed region to be proximate to the first engaging surface of the first end of the mid region of the intermedullary device. The method comprises causing at least a portion mid region of the intermedullary device to be configured to be proximate to at least a portion of the second exposed region after having been provided in the second medullary canal of the distal portion of the bone in a manner for cortical bone at or near the second exposed region to be proximate to the second engaging surface of the second end of the mid region of the intermedullary device.

In one or more embodiments is a method of fixating a bone with an intermedullary device, in which the bone comprises a proximal portion having a first medullary canal and a first exposed region, and a distal portion having a second medullary canal and a second exposed region. The method comprises providing a first end of the intermedullary device in a first medullary canal at the first exposed region, the first exposed region being at or near a mid portion of the bone, the intermedullary device comprising a mid region and a cross-sectional diameter in the mid-region being at least 10% greater than any other cross-sectional diameter of the intermedullary device that provides a first engaging surface at a first end of the mid region of the intermedullary device and provides a second engaging surface at a second end of the mid region of the intermedullary device. The method comprises providing a second end of the intermedullary device in a second medullary canal at the second exposed region. The method comprises causing at least a portion of the mid region of the intermedullary device to abut at least a portion of the first exposed region after having been provided in the first medullary canal of the proximal portion of the bone in a manner for cortical bone at or near the first exposed region to be proximate to the first engaging surface of the first end of the mid region of the intermedullary device. The method comprises causing at least a portion mid region of the intermedullary device to be proximate to at least a portion of the second exposed region after having been provided in the second medullary canal of the distal portion of the bone in a manner for cortical bone at or near the second exposed region to be proximate to the second engaging surface of the second end of the mid region of the intermedullary device.

In one or more methods, configuring the first end of the intermedullary device for the first medullary canal at the first exposed region may include configuring the first end to extend to a length as to be proximate to a diaphyseal region of the proximal portion of the bone without being of a length to penetrate the diaphyseal region of the proximal portion. In the method, configuring the second end of the intermedullary device for the second medullary canal at the second exposed region may include configuring the first end to extend to a length as to be proximate to a diaphyseal region of the distal portion of the bone without being of a length to penetrate the diaphyseal region of the distal portion. In one or more methods, configuring at least a portion of the mid region of the intermedullary device to abut at least a portion of the first exposed region may include configuring at least a portion of the first engaging surface of the first end of the mid region of the intermedullary device for abutting cortical bone of the first exposed region. In one or more methods, configuring at least a portion mid region of the intermedullary device to be proximate to at least a portion of the second exposed region may include configuring at least a portion of the second engaging surface of the second end of the mid region of the intermedullary device for abutting cortical bone of the second exposed region.

In one or more methods, providing a first end of the intermedullary device in the first medullary canal at the first exposed region includes extending the first end of the intermedullary device so it is at or is proximate to a diaphyseal region of the proximal portion of the long bone without penetrating the diaphyseal region of the proximal portion. In one or more methods, providing the second end of the intermedullary device in the second medullary canal at the second exposed region includes extending the second end of the intermedullary device so it is at or is proximate to a diaphyseal region of the distal portion of the long bone without penetrating the diaphyseal region of the distal portion. In one or more methods, causing at least a portion of the mid region of the intermedullary device to abut at least a portion of the first exposed region may include causing at least a portion of the cortical bone to abut the first engaging surface of the first end of the mid region of the intermedullary device. In one or more methods, causing at least a portion mid region of the intermedullary device to be proximate to at least a portion of the second exposed region may include causing a portion of the cortical bone to abut the second engaging surface of the second end of the mid region of the intermedullary device. Any of said methods may further comprise locking the intermedullary device at the mid region without any external fixation elements and without any separate anchoring elements.

Any of said methods described herein may further comprise configuring the intermedullary device for locking with the bone without any external fixation elements and without any separate anchoring elements.

These and additional embodiments are further described below.

### BRIEF DESCRIPTION OF THE DRAWINGS

Various embodiments will be explained in more detail with reference to the drawings in which:

FIG. 1 depicts a first portion of the device described herein;

FIG. 2 depicts a second portion of the device described herein;

FIG. 3A depicts a schematic of a representative device described herein;

FIG. 3B depicts a schematic of another representative device described herein;

FIG. 3C depicts a schematic of another view of the representative device of FIG. 3B;

FIG. 3D depicts a schematic of a cross section of the representative device of FIG. 3B taken along a plane formed by line Y of FIG. 3B;

FIG. 4 depicts another representative device described herein;

FIG. 5 depicts still another representative device described herein;

FIG. 6 depicts a radiograph of a representative device described herein installed in the medullary canal of a bone;

FIG. 7 depicts a further representative device described herein;

FIG. 8 depicts yet another representative device described herein;

FIGS. 9A and 9B illustrate representative surgical instruments developed for use with the device described herein;

FIGS. 10A to 10O illustrate representative steps in a process described herein for installing one of the devices described herein;

FIG. 11 illustrates a representative radiograph image of a representative device described herein when installed as described herein, in which the representative device is indicated by the arrow;

FIGS. 12 A, C, and E illustrate representative radiographs of the representative device described herein at day 7, day 14 and day 21 post installation;

FIGS. 12 B, D, and F illustrate close-ups of the radiographs of FIGS. 12A, C, and E, respectively, showing the defect region;

FIG. 13 illustrates a longitudinal scan of the device installed in the bone of FIG. 12;

FIGS. 14 to 16 illustrate cross-sectional reconstructions made using the scan of FIG. 13;

FIG. 17 depicts representative volumetric measurements of new bone at day 7, 14 and 21 post installation of a device described herein (n=3);

FIG. 18 depicts representative polar moment of inertia measurements obtained at three axial sections (n=3);

FIG. 19 depicts a representative coronal cross section of device described herein 1 day after installation in the long bone of a mammal; and

FIG. 20 depicts a longitudinal section outlined by the box in FIG. 19, the section further stained histologically to show bone outgrowth.

### DESCRIPTION

Although making and using various embodiments are discussed in detail below, it should be appreciated that as described herein are provided many inventive concepts that may be embodied in a wide variety of contexts. Embodiments discussed herein are merely representative and do not limit the scope of the invention.

The devices described herein may be utilized for stabilization and/or fixation of bone having a medullary canal. Many current intermedullary devices for stabilization and fixation of bone lack both axial and rotational stability; they also lead to a high risk of dislocation. The devices described herein help overcome these issues when installed in the bone of a mammal, such as long bone, which includes but is not limited to the femur, tibia, fibula, humerus, radius, ulna, metacarpal, metatarsal, phalange, and clavicle.

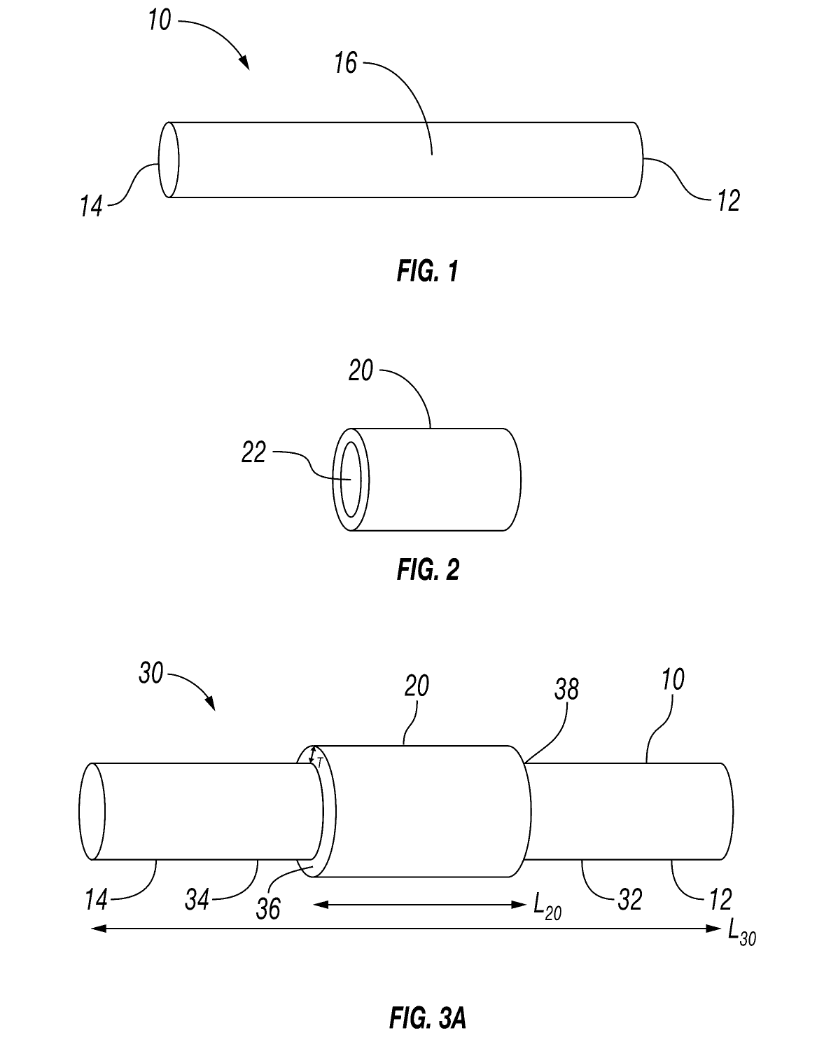

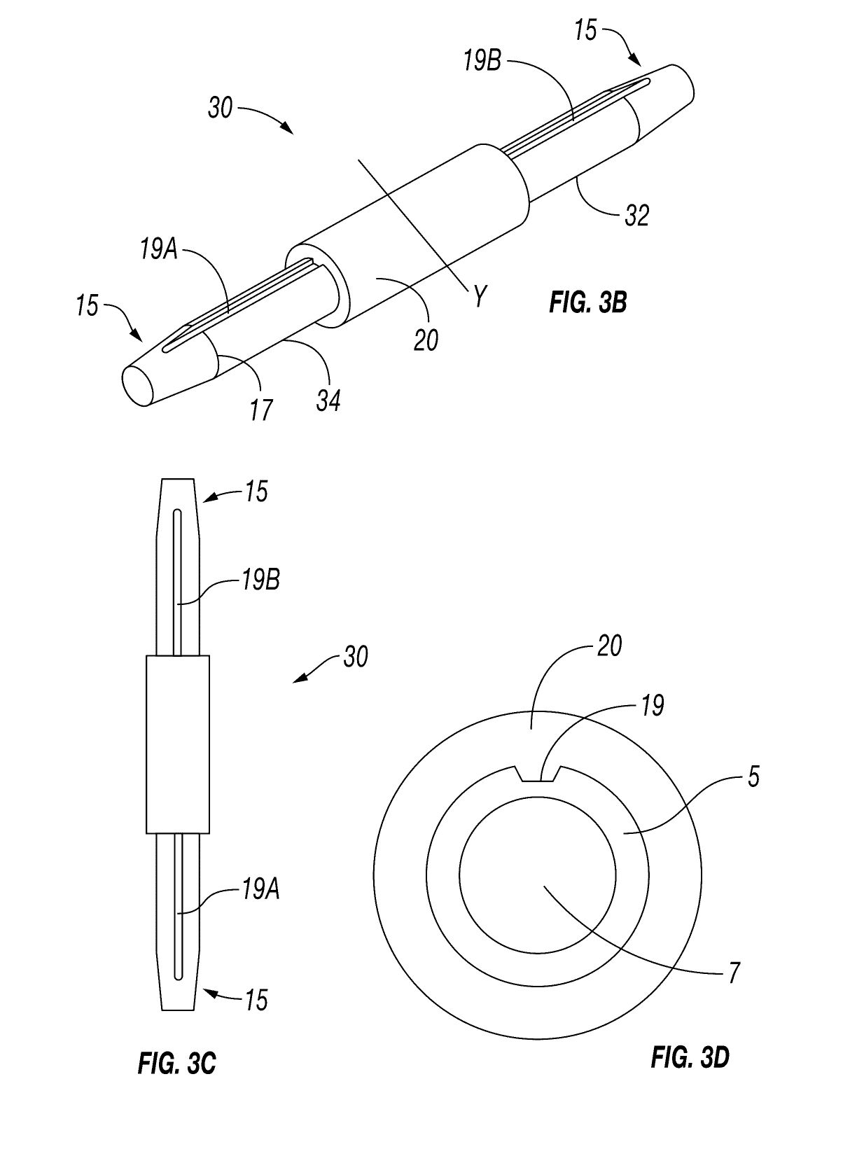

Referring first to FIGS. 3A, 3B, 3C, and 4-8, various representative embodiments of intermedullary device 30 as described herein are illustrated. Said devices generally include a first portion and a second portion, each of the first portion and/or the second portion may include one or more features provided independently and/or formed integrally. Said devices may include a first portion and a second portion, in which the first portion and the second may further include one or more features, and the first portion and the second portion may be provided independently and/or may be formed integrally. The first portion is an elongate member 10 as depicted at least in FIG. 1. The elongate member has first and second opposing ends 12, 14 and a mid-region 16. Elongate member 10 may be synthesized and/or formed from a thermoplastic or thermoset resin, one that forms a plastic material offering sufficient mechanical strength and rigidity for stabilization of the long bone. The material may be plastic or may be a composite, a long as the material has sufficient strength and rigidity for stabilization of the bone. The plastic material is one that is non-toxic (e.g., in a biologic system). The composite material should be a material that is non-toxic in a biologic system. The plastic material is preferably one that does not induce an inflammatory response (e.g., in a biologic system). The composite material is preferably one that does not induce an inflammatory response (e.g., in a biologic system). In some embodiments, however, the material may be selected to induce inflammation or an inflammatory response. And, in some embodiments, the material may be selected as one that is toxic. Hence, it may be that in some embodiments, the specific material may be selected as one that illicits one or more biologic responses to a toxic material and/or to an inflammation-inducing material. In one or more embodiments, the elongate member is synthesized and/or otherwise fabricated from a plastic material that is formable to the shape of the elongate member. In one or more embodiments, the elongate member is synthesized and/or otherwise fabricated from a composite material that is formable to the shape of the elongate member. In one or more embodiments, the elongate member is synthesized and/or otherwise fabricated from such a plastic material that is formable and, after forming said shape, may be further shaped (e.g., milled, or micromilled for very small devices) for more precise fitting. In one or more embodiments, the elongate member is synthesized and/or otherwise fabricated from such a material that is formable to a first shape, and, after forming said first shape, may be further formed to a second shape (e.g., milled, or micromilled for very small devices), the second shape providing more precise fitting.

A representative example of a suitable plastic material for the elongate member 10 is polyether ether ketone (PEEK), which is a semicrystalline thermoplastic that can be molded, and may be further shaped after molding (e.g., by milling). The elongate member 10 may also be comprised of a polyaryletherketone plastic. The elongate member 10 may also be comprised of a polycarbonate plastic. The elongate member 10 may also be comprised of any of a family of polyfunctional methacrylate resins including one or more dimethacrylate monomers and mixtures thereof (e.g., bis-phenol and glycidyl methacrylate or Bis-GMA, urethane dimethacrylate or UDMA, 1,6-hexanediol dimethacrylate or HDDMA, triethylene glycol dimethacrylate or TEGMA, methacrylate-thiol-enes, ethoxylated bisphenol A dimethacrylate or EBPADMA) with or without fillers (e.g., crystalline silicate particles, aluminosilicate particles, borosilicate particles, zirconium ions, zinc, barium, etc.). The elongate member 10 may also be comprised of a bioplastic, such as ones containing polylactic acids, hot-pressed cellulose hydrogels, or poly hydroxybutyrate biopolyester, to name just a few. The one or more plastics may be reinforced with fibers, such as carbon fibers, and/or glass fibers. The elongate member 10 may also be comprised of porcelain. The elongate member 10 may also be comprised of a metal, such as titanium alloy, or zirconium, or lithium. For example, the material may be impregnated with one or more metal salts and/or electrolytes. The metal salts and/or electrolytes may be one or more that enhance osteointegration and/or blood coagulation (e.g., lithium salt, silicon nitride, ortho-phosphoric acid, sulphuric acid, fluoride salt). However, metals that scatter x-ray, such as steel and titanium, are generally not preferred. In one or more embodiments, metals that scatter x-ray may be considered unsuitable as a material for the devices described herein. Similarly, plastics that are considered highly flexible with low strength, such as polystyrene or polypropylene, are also generally considered unsuitable or less suitable as a material for the devices described herein. Composites or combinations of the described materials or other materials that meet the needs described herein for forming the elongate member may also be used to form the elongate member 10.

Generally, the elongate member 10 will not include bores or anchoring holes since the devices 30 described herein do not require extra fixation (either by external fixation, or with additional screws, locking plates, etc.). The elongate member 10 will often have a circular cross-section, as depicted in FIG. 1. However, alternative shapes may be used (e.g., polygon, ellipse, or some combination thereof). In some embodiments, the elongate member may comprise an outer layer 5 over an inner core 7, as depicted in FIG. 3D, which is a cross section of a mid-region of an elongate member 10, further illustrated with a collar or band 20, which is described below. The dual nature, as depicted in FIG. 3D, may enhance strength (e.g., tensile and/or shear) of the elongate member 10. The dual nature, as depicted in FIG. 3D, may enhance strength (e.g., tensile and/or shear and/or bending) of the elongate member 10. The dual nature, as depicted in FIG. 3D may provide a cost savings and/or allow use of a stronger material in one layer. The dual nature, as depicted in FIG. 3D may allow use of a more toxic material in the inner core 7. One of the inner core 7 or the outer layer 5 may be made of a stronger and/or tougher material. In some embodiments, the elongate member 10 may comprise a tapered end or tapered portion 15 for one or both of the first and second opposing ends 12, 14. Such a tapering is depicted in FIGS. 3B, and 3C. Said end or portion, when tapered may retain the same general or overall shape, though having a smaller cross sectional diameter. Said end or portion, when tapered may, instead, form a different general shape. The tapered portion 15 may be integral with the elongate member. In one or more embodiments, the tapered portion 15 may be of the same material as the elongate member. In one or more embodiments, the tapered portion 15 may be of a different material than the elongate member. In one or more embodiments, the tapered portion 15 may be beveled to form the tapered portion 15. In one or more embodiments, the tapered portion 15 may be fitted to an end of the elongate member 10, such as via a snap fit, a screw, and/or press fit. An example is depicted in FIG. 3B. In one or more embodiments, the tapered portion 15 may provide a seam or line 17 between the beveled or tapered portion 15 and the elongate member 10, such as depicted in FIG. 3B. In some embodiments, the elongate member 10 may comprise ridges, grooves and/or one or more extending features at or near one or both of the first and second opposing ends 12, 14. In addition or as an alternative, in one or more embodiments, the elongate member 10 may comprise ridges, grooves and/or one or more extending features at or near the mid-region 16. In addition or as an alternative, in one or more embodiments, the elongate member 10 may comprise ridges, grooves and/or one or more extending features that run along a length of the elongate member 10, as depicted in cross section in FIG. 3D, which shows a groove 19. In one or more embodiments, the one or more ridges, grooves and/or one or more extending features fun along a length of the elongate member 10 from a mid-region to proximate the end of the elongate member. An example of this is depicted in FIGS. 3B and 3C, showing a first groove 19A and a second groove 19B. In the illustrations, the first and second grooves 19A and 19B extend along a length of the elongate member 10 and through to the beveled or tapered portion 15. Alternative lengths are understood to be acceptable and/or utilized. In some embodiments, the elongate member 10 may further comprise a textured surface, e.g., roughening, grooves, tabs, barbs, such as near the mid-region 16 or near one or more of first or second opposing ends 12, 14. In addition or as an alternative, the elongate member may further comprise a coating on some or all of its exposed surface or outer surface. The coating may comprise a thin coating, as a surface layer or as a lamina (more than one layer), and may or may not be and/or contain therein one or more biocompatible materials, such as one or more biodegradable materials, cells, proteins, peptides, hormones, growth factors or other biologic factors or biologic compounds, or materials having or exhibiting bioactivity. Thus, when introduced and/or utilized, the one or more biocompatible materials may be on the surface of the device, or may be intermixed within a surface coating of the device, or may be throughout all or most of a matrix forming one or more coatings of the device. The one or more coatings may be introduced to the elongate member 10 by spraying, dipping, pouring, painting, or 3D printing, as representative examples, knowing that alternative means known in the art or later developed may also be utilized. The one or more coatings when provided may further include one or more drying, curing and/or hardening components and/or steps.

Referring to FIG. 2, there is depicted schematically a second portion of device 30. The second portion is a collar or band 20. The band 20 will have a hollowed center 22. In some embodiments, the band 20 is comprised of the same material or similar material as the elongate member 10. In some embodiments, the band 20 is comprised of a different material than the elongate member 10. The material of band 20 is preferably compatible with, and/or capable of contacting, and/or engaging with, and/or bonding with the material of elongate member 10, or at least with the outer material of the elongate member 10. A cross section of band or collar 20 contacting and/or engaging with elongate member 10 is depicted schematically in FIG. 3D. The band 20 generally comprises a material that has sufficient compressive strength. In one or more embodiments, the sufficient compressive strength is one that prevents bone to bone contact when the device is utilized, such as in a manner as described herein. In one or more embodiments, the band 20 should be comprised of material that provides both strength and rigidity. In some embodiments, the band 20 may further comprise a textured surface, e.g., roughened, grooved, barbed, tabbed, and/or having a porosity. In some embodiments, band 20 is further coated with a coating, a surface layer, or a lamina (two or more layers), as described above. In one or more embodiments, the coating on band 20 is the same as the coating on elongate member 10, when elongate member 10 is coated. In one or more embodiments, the coating on band 20 is different than the coating on elongate member 10, when elongate member 10 is coated. Thus, elongate member 10 and/or band 20 may comprise and/or may independently comprise one or a plurality of coatings thereon. Said coatings may include one or more biocompatible materials, such as one or more biodegradable materials, cells, proteins, hormones, growth factors, and/or other biologic factors, and/or biologic compounds or materials having or exhibiting bioactivity. The one or more biocompatible materials may be on the surface, or intermixed within the surface coating, or may be throughout all or most of a matrix formed by said coating. Similar to when coating the elongate member, the coating when provided to the band 20 may further include one or more steps of drying, curing, and/or hardening, with the appropriate components for drying, curing and/or hardening.

The band 20 may, in some embodiments, be formed independently from the elongate member 10. For example, the elongate member 10 is provided (e.g., as one unit, or as having an outer layer 5 over an inner core 7), and it may be molded, and/or milled, cut or further sized. In one or more embodiments, the band 20 is formed or otherwise prepared independently and then passed over the elongate member 10, thereby forming device 30, such as depicted in FIG. 3A. Here, band 20 includes the hollowed center 22 prior to passing over the elongate member 10. And here, band 20 will generally have sufficient compressive strength to prevent defect damage and/or narrowing of the defect size when utilized in a manner such as described herein. And, band 20 may be further engaged with, and/or bonded with, and/or interconnected with, and/or joined with elongate member 10 to ensure that band 20 does not move, or become displaced, once positioned on elongate member 10. The joining or bonding may comprise a bonding agent or adhesive or chemical material or layer therebetween. The joining or bonding may comprise a tight fitting. The joining or bonding may comprise suitable means for joining or bonding, which may include a fastening, crimping, grooving, roller expanding, swaging, as representative examples, or alternative means known in the art. Alternatively, or in addition, bonding may comprise one or more of drying, heating, irradiating, soldering, welding or the like, as representative means of joining. In one or more embodiments, bonding or joining may comprise an agent or glue in order to adhere band 20 with elongate member 10. In this embodiment, some further means or material for bonding or joining may be necessary. Band 20 is generally positioned on all or a portion of mid-portion 16 of elongate member 10, so that device 30 will include proximal region 32 and distal region 34, such as depicted in FIGS. 3A, 3B, and 4-6. The proximal region 32 and distal region 34 may have similar lengths, or proximal region 32 and distal region 34 may differ in length, depending, in part, on the bone, and/or a location of the bone defect, and/or on design choice, as examples.

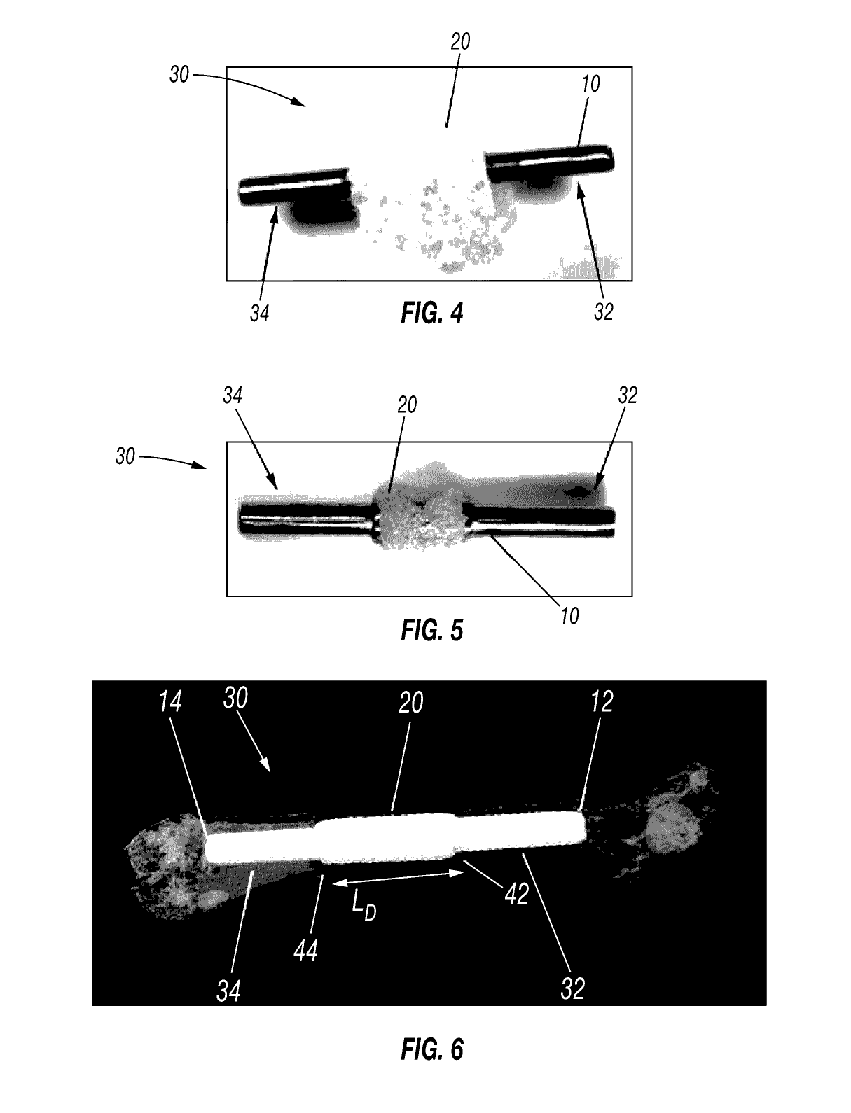

In another embodiment, the band 20 may be comprised of the same material or may be of a similar or different material as the elongate member 10, and is formed integral with the elongate member 10. For example, the elongate member 10 and the band 20 are provided (e.g., the elongate member 10 provided as one unit, or having an outer layer 5 over an inner core 7), such as by molding as a unit, and/or milling from a material, and may be further cut or further sized. In another example, the elongate member 10 is provided (e.g., as one unit, or as having an outer layer 5 over an inner core 7), it may be molded and/or milled, cut or further sized. The band 20 is formed or otherwise prepared on the elongate member 22, thereby forming device 30, such as depicted representatively in FIGS. 4 and 5. With reference to FIGS. 4 and 5, band 20 is formed or otherwise prepared as one or more coating layers, being any one or combination of a unified material, composite, dispersion, foam, particulate, emulsion, droplet, or the like, which may be sprayed, dipped, poured, painted, or 3D printed, as representative examples of means for forming and applying or coating band 20 on the elongate member 10. Band 20 may include a first or joining or bonding layer that may be the same or different than one or more subsequent layers. The first or joining layer may comprise a bonding agent or adhesive material. Alternatively, or in addition, joining of band 20 with elongate member 10 may include heating, drying, sonicating, irradiating, and/or curing, as representative means to ensure that band 20 is adhered or otherwise joined with elongate member 10. The means or step of joining or bonding may be performed at the same time, or subsequent, to forming and/applying band 20. The one or more bands 20 may be joined, formed, and/or applied to elongate member 10. The one or more bands may be the same or different. In addition, or as an alternative, one or more coatings may be joined, or applied or formed with band 20 or to the elongate member 10. The one or more coatings may be the same or different. At least some of the material used to form band 20 will have sufficient compressive strength to prevent defect damage and/or narrowing of the defect size. In such embodiments, such as depicted in FIGS. 4 and 5, band 20 is generally positioned on or near a mid-portion 16 of elongate member 10, and device 30 will include proximal region 32 and distal region 34, such as depicted in FIGS. 4 and 5. The proximal region 32, and the distal region 34 may have similar lengths, or may differ in length, depending, in part, on the bone, and/or a location of the bone defect, and/or on design choice, as examples.

In a further embodiment, at least a portion of band 20 is comprised of the same material as the elongate member 10 and formed integral with the elongate member 10. For example, the elongate member 10 when formed will include some or all of band 20 extending therefrom. Here, the elongate member 10 with at least some of band 20 is formed by molding, and/or by milling (e.g., the elongate member 10 provided as one unit, or as having an outer layer 5 over an inner core 7). Thus, at least some of band 20 will have sufficient compressive strength to prevent defect damage and/or narrowing of the defect size. Band 20 and/or elongate member 10 may be complete upon molding, or may be provided in final form by further milling, carving, and/or by further coating, thereby forming device 30 having proximal region 32 and distal region 34, such as depicted in FIG. 6. Here, any one or more of band 20, proximal region 32, and/or distal region 34 may include a coating, the coating being any one or the coating forms described above (e.g., unified material, composite, dispersion, foam, particulate, emulsion, droplet, etc.), provided by any of the means described above (e.g., spraying, dipping, pouring, painting, 3D printing, etc. with or without heating, drying, sonicating, irradiating, and/or other means for curing and/or hardening). Band 20 may include a first or bonding layer on its exterior surface prior to coating or completing the band 20. The first or bonding layer may be the same or different than one or more subsequent layers. The first or bonding layer may comprise a bonding agent or adhesive material. Any further layer applied to band 20 may require additional bonding, heating, sonicating, actinic radiation, and/or curing, as representative means to ensure that the subsequent layers adhere to band 20. Similarly, proximal region 32 and/or distal region 34 may include one or more layers thereon. Here, the means or step of providing, forming, and/or bonding the one or more external layers may be performed at the same time or may be performed after forming (e.g., molding and/or milling, and/or carving, and/or sizing). In this embodiment, band 20 is generally positioned about the mid-portion 16 of elongate member 10. In some embodiments, the proximal region 32 and distal region 34 have similar lengths. In some embodiments, the proximal region 32 and distal region 34 will differ in length. As before, positioning of the proximal region 32 and distal region 34 will depend, in part, on location of the bone defect, or on design choice, as examples.

Device 30 when formed will include, generally in its mid region, band 20 having, on average, a larger cross-sectional diameter than any cross-sectional diameter found along proximal region 32 and distal region 34 of the elongate member 10. The larger cross-sectional diameter of band 20 (or at least a portion thereof) may be up to 10% greater, or about 10% greater, or more than 10% greater than a cross-sectional diameter of any of the proximal region 32 of the elongate member 10, or the distal region 34 of the elongate member 10. The larger cross-sectional diameter may, in some embodiments, not exceed 10% of the cross-sectional diameter of any of the proximal region 32 or the distal region 34. In some embodiments, the band 20 may comprise variations in the cross-sectional diameter. Generally, in such cases, a least a portion of the band will have a cross-sectional diameter that is greater than the cross-sectional diameter of the elongate member 10. In some embodiments, the band 20 may comprise a stiff and rigid portion along with a softer and/or less rigid and/or less stiff portion. This may occur, for example, when the band 20 comprises a plurality of bands, as depicted in FIG. 8, as band 20a and band 20b, or may when the band 20 comprises a plurality of bands, such as one or more outer bands and one or more inner band, some of which are more or less rigid. This may occur, for example, when the band 20 comprises two materials (e.g., two layers), and/or when the band 20 comprises at least one body portion and at least one coating. In one or more embodiments, the body portion (that having the more rigid and stiff material) may be about or less than about 10% greater that the cross-sectional diameter of the proximal region 32 or the distal region 34. In some embodiments, the body portion may be about or less than about 15% greater than the cross-sectional diameter of the proximal region 32 or the distal region 34, or may be about or less than about 20% greater than the cross-sectional diameter of the proximal region 32 or the distal region 34.

The cross sectional diameter of the band 20 may also be up to, at, or about 15% greater than the cross-sectional diameter of any of the proximal region 32 and/or the distal region 34 of device 30. The cross sectional diameter of the band 20 may be up to, at, or about 20% greater than the cross-sectional diameter of any of the proximal region 32 or the distal region 34. The cross sectional diameter of the band 20 may be up to, at, or about 25% greater than the cross-sectional diameter of any of the proximal region 32 or the distal region 34. The cross sectional diameter of the band 20 may also be up to, at, or about 30% greater than the cross-sectional diameter of any of the proximal region 32 or the distal region 34. The cross sectional diameter of the band 20 may be up to, at, or about 50% greater than the cross-sectional diameter of any of the proximal region 32 or the distal region 34. Generally, the cross sectional diameter of the band 20 may be in any range from about 5% greater and up to or at about 60% greater than the cross-sectional diameter of any of the proximal region 32 or the distal region 34. While larger cross-sectional diameters are possible, they are not often provided unless the band comprises a body portion (having the more rigid and stiff material) and one or more coating portions (comprising the softer, less rigid and/or less stiff material). In some embodiments, larger cross-sectional diameters of band 20 are not cost-effective. In addition, the greatest cross-sectional diameter of band 20 (with may be all of band 20 or only a portion of band 20) will not generally be greater than the average cross-sectional diameter of the mid diaphyseal region of the long bone to which the device 30 is fitted for. In one or more embodiments, an average cross-sectional diameter of the mid diaphyseal region of bone may be utilized to define a maximal cross-sectional diameter of the band 20. In one or more embodiments, an average cross-sectional diameter of a medullary canal may be utilized to define a maximal cross-sectional diameter of the elongate member 10. In one or more embodiments, an average cross-sectional diameter of a proximal region of a medullary canal may be utilized to define a maximal cross-sectional diameter of the proximal region 32 of the elongate member 10. In one or more embodiments, a cross-sectional diameter (or an average cross-sectional diameter) of a proximal end (or a region proximate to the proximal end) of a diaphyseal region of the medullary canal may be utilized to define a maximal cross-sectional diameter of the proximal region 32 of the elongate member 10. In one or more embodiments, an average cross-sectional diameter of a distal region of a medullary canal may be utilized to define a maximal cross-sectional diameter of the distal region 34 of the elongate member 10. In one or more embodiments, a cross-sectional diameter (or an average cross-sectional diameter) of a distal end (or a region proximate to the distal end) of a diaphyseal region of the medullary canal may be utilized to define a maximal cross-sectional diameter of the distal region 34 of the elongate member 10.

Proximal region 32 of device 30 will extend to first opposing end 12 while distal region 34 will extend to and end at second opposing end 14 (see, e.g., FIG. 3A, FIG. 6). Importantly, by the configurations described herein, device 30 will include on one side of band(s) 20 a lip 36, which is proximate to and/or capable of abutting a first side edge of bone that is on the distal end of the defect site (see defect end or exposed end 44 of FIG. 6), and on an opposing side of band 20 there is lip 38, which is proximate to and capable of abutting a second side edge of bone that is on the proximal end of the defect site (see defect end or exposed end 42 of FIG. 6). The thickness (T) of lips 36 and 38 may be up to, or at least, or about half the thickness of the cortical bone of the long bone in which the device 30 is to be installed (see FIG. 3A; see also FIG. 6). In some embodiments the lips 36 and 38 are at least or about 25% of the thickness of the cortical bone of the long bone. The lips 36 and 38 do not need to have identical thicknesses. The lips 36 and 38 may also be greater than 25% or greater than 50% the thickness of the cortical bone. Lip 36 and/or lip 38 may further comprise ridges, grooves, latticework, and/or one or more biocompatible features, as examples, with or without one or more bioactive agents or compounds.

In one or more embodiments, the cross sectional diameter of the proximal region 32 and distal region 34 of device 30 will generally be about the same. This occurs for bone having a similar or substantially similar cross sectional area of the medullary canal, when measured at some or several spaced apart regions along the diaphyseal region, and/or when measured at some or several regions spaced apart from the mid diaphyseal region. In one or more embodiments, the cross sectional diameter of the proximal region 32 and distal region 34 of device 30 may differ. This may occur for bone having dissimilar cross sectional areas of the medullary canal, when measured at some or several spaced apart regions along the diaphyseal region, and/or when measured at some or several regions spaced apart from the mid diaphyseal region. In one or more embodiments, said cross sectional diameters will be about or only slightly less than the cross-sectional diameter of the intermedullary canal of the long bone of the mammal into which the device is configured for, and/or is to be installed. The diameter of the intermedullary canal of the long bone of the mammal may be precisely identified by the means, methods and systems described herein, which may include sizing the device based on one or a plurality of scans (e.g., from a population of the mammal, which may also be further categorized based on age, and/or weight, and/or sex of the mammal), and/or may include three-dimensional reconstructions of the long bone of the mammal (which may also be further categorized based on age, and/or weight, and/or sex of the mammal). In one or more embodiments, said cross sectional diameter of the proximal region 32 and the distal region 34 of device 30 should provide an interference fit when installed in the medullary canal of a bone. In one or more embodiments, having a more precise cross sectional diameter of the proximal region 32 and distal region 34 of the device 30, in which the cross sectional diameters are based on the long bone of the mammal into which the device is to be installed, provides an interference fit when installed. The interference fit is one means by which the device 30 described herein may be installed with addition fixation. The interference fit also reduces translational movement of the device 30 after installation. The interference fit is generally obtained by having the cross-sectional diameter of the proximal region 32 and distal region 34 of device 30 within 5%, or within 10%, or within 15%, of the cross-sectional diameter of the intermedullary canal of the long bone of the mammal into which the device is to be installed. The interference fit may be obtained by having the cross-sectional diameter of the proximal region 32 and distal region 34 of device 30 not more than 20% different than the cross-sectional diameter of the intermedullary canal of the long bone of the mammal into which the device is to be installed.

The length of band 20 (L₂₀) may be as much as one-third, or about one-third of the length of the device 30 (L₃₀) (see FIG. 3A). The length of band 20 may also be smaller. The length of band 20 may also be larger than one-third the length of the device 30. The length of band 20 generally does not comprise more than 50% or 60% the length of the device 30. In many embodiments, the length of band 20 is sized to be about the same length as the size (length) of the defect (L_{D} in FIG. 6). For example, for a female nude (Nu/J) mouse, 6 weeks of age (e.g., 18-25 pounds), the defect size may be about 3 mm, and will be made in the mid diaphyseal region (see, e.g., FIG. 6). Accordingly, the suitable length of band 20 for device 30 will generally be about 3 mm. In this example, the entire length of device 30 (L₃₀) may be 9 mm, which is sufficient for proximal region 32 and distal region 34 to reside and extend within the medullary cavity, along the diaphyseal region without penetrating or damaging the metaphysis. Thus, device 30 will often have a length that is about the same or just slightly smaller than the length of the diaphyseal region of the long bone into which the device 30 is to be fitted. In this example, the band was about 33% or ⅓ the full length (L₃₀) of the device 30. This is only representative. The band 20 may be less than ⅓ the full length (L₃₀) of the device 30. The band may be about 30% the full length (L₃₀) of the device 30, or may be about 25% the full length (L₃₀) of the device 30, or may be about 20% the full length (L₃₀) of the device 30, or may be about 15% the full length (L₃₀) of the device 30, or may be about 10% the full length (L₃₀) of the device 30, or may be about 5% the full length (L₃₀) of the device 30, or may be any value therebetween. While larger lengths for band 20 are also possible (e.g., greater than 33% of the total length (L₃₀) of the device 30, or greater than 35% of the total length (L₃₀) of the device 30, or greater than 40% of the total length (L₃₀) of the device 30, etc), for purposes of research and investigation, they are not as practical.

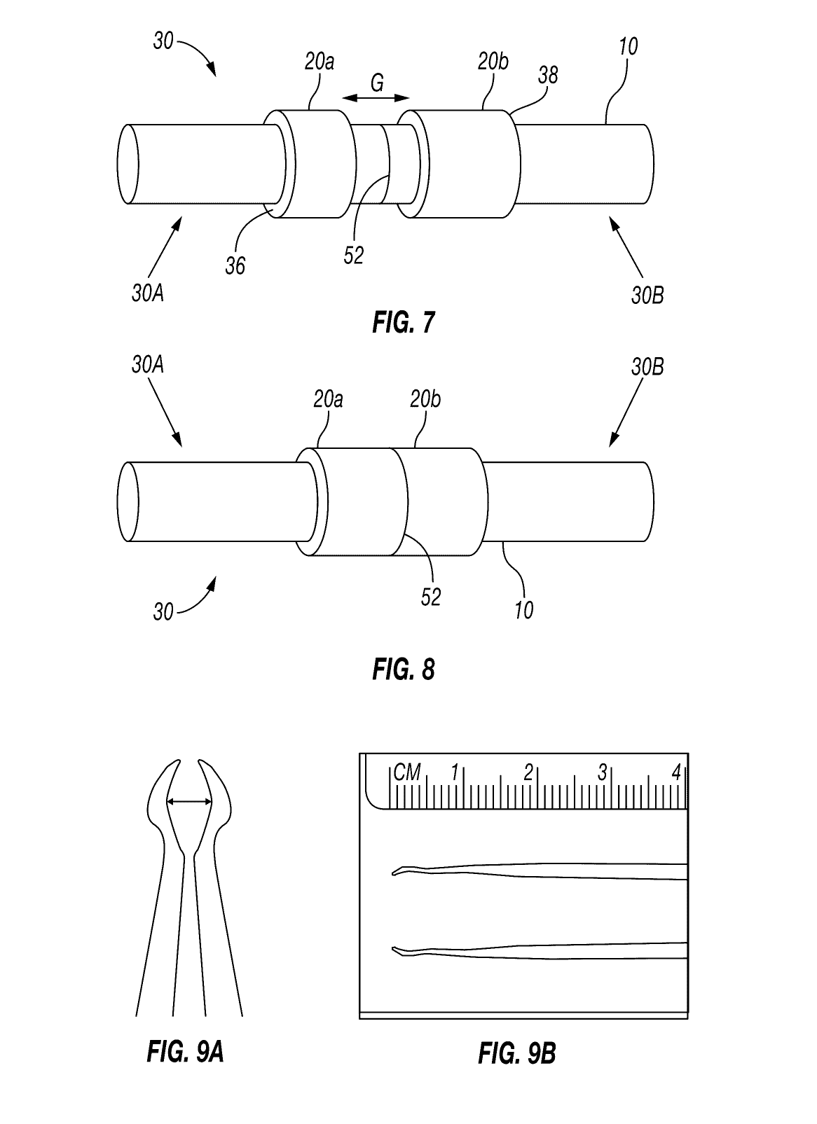

In some embodiments, device 30 may comprise two or more bands 20 positioned in series. The bands may be contiguous, such as depicted in FIG. 8, or may be spaced apart and having a gap (G) therebetween, such as depicted in FIG. 7. Said two or more bands may be formed as described above. With at least two bands, at least one of the bands, depicted in FIG. 7 as band 20a, will comprise a lip 36, which is proximate to and capable of abutting a first side edge of bone that is on the distal end of the defect site, and at least one separate band, depicted in FIG. 7 as band 20b, will comprise a lip 38, which is proximate to and capable of abutting a second side edge of bone that is on the proximal end of the defect site.

Device 30 may also, in one or more embodiments, comprise two segments that couple. The means for coupling or joining are any known means for mating and/or interlocking a first segment 30A and a second segment 30A at a joint 52 as is known in the art, such as depicted in FIGS. 7 and 8. Preferably, the first and second segments 30A and 30B are fully joined. Preferably, when the first and second segments 30A and 30B are fully joined, there is no further rotational and translational movement between or with respect to the two segments. Accordingly, the joint 52, regardless of the type of joint, will generally include at least one locking means and/or mechanism for securely and fixedly engaging the first segment with the second segment. It is understood that the joint 52 may also be positioned at any point along elongate member 10, and may be away from or removed from the band 20. Joint 52 may assist in positioning the device 30 when it is installed in the medullary canal or medullary cavity of the long bone.

Device 30, as described and as represented at least in FIGS. 3A, 3B, 3C and 4-8, unlike alternative intermedullary devices, prevents slippage when positioned in the medullary canal of the mammal. Slippage is especially problematic with other intermedullary devices that are not properly dimensioned, or are not further screwed, or are not otherwise fixed after they are installed in the medullary canal. In particular, with the device described herein, the addition of band 20, having a cross-sectional diameter that is larger than any cross-sectional diameter found on proximal region 32 or distal region 34, provides proximate co-location and/or abutment of band 20 with at least a portion of first and second bone ends 42, 44 at the defect site, preventing defect damage as well as translation of device 30 along or around the defect, and narrowing and/or significant translation of the defect itself is also avoided (see e.g., FIG. 6). With the device described herein, the proximal region 32 and distal region 34 are accurately and/or precisely sized, as described further below, in order to eliminate damage to the growth plate, metaphysis, and distal epiphysis of the long bone. Furthermore, said device 30 is introduced or installed in the medullary canal of the long bone without requiring surgical excisions to or for entry through the metaphysis or distal epiphysis of the long bone.

The device 30 may be scaled to very small sizes and is particularly advantageous for use in mice, which have proven to be problematic when attempting to use other (or alternative) intermedullary devices, due to the very small size of their bones, including long bones. The device 30 being scalable to very small sizes for use in very small mammal, such as mice, affords several unique advantages because mice, for example, undergo rapid repair and remodeling to allow subsequent events and/or results to be monitored very quickly. And, certain mice have been developed with specific strains that do not reject human proteins or cells, or have been developed with a number of transgenic models that allow for very targeted research and analysis, and require only a small fraction of the test materials needed when using larger mammal (and at much reduced cost).

In one or more embodiments, the device 30 described herein will include suitable and discernible markers when they are formed. Said markers may be imprinted or otherwise introduced to the device in order to follow progress during the repair and remodeling process. Said markers may include specific dyes and/or colorants that will appear during one or more scannings, radiographs or other means used to follow progress during repair and remodeling of bone. The device 30 may include one or more metal markers or micro-particles at one or a number of various landmarks on the pin, said markers being suitable for radiography and/or for observational purposes. For example, the plastic material of proximal region 32 and/or the distal region 34, and/or said coating material thereon, may be impregnated at one or more specific locations with a fluorescent dye that permits visualization of the device 30 through skin and muscle, but not bone tissue. With such embodiments, live animal fluorescence may be utilized to monitor healing without the need for alternative visualization methods, such as an x-ray system. The one or more markers though not shown may be provided on band 20, and/or on one or both of the proximal region 32 and/or the distal region 34. In one or more embodiments, at least one or more markers are usually at least near the defect end 42 and/or the defect end 44 as depicted in FIG. 6.

The device 30 may be sized and shaped in advance by mapping long bone lengths, medullary canal diameters, diaphysis lengths and cortical thicknesses for each mammal strain, and may be further mapped by sex, by sex and age, by sex and age and weight, or any combination thereof. Suitable ages for mapping one or more long bones of smaller mammals may be from about three to four weeks to about 12 weeks or older. The mapping may include initially scanning the long bone, such as with a microCT scanner, to provide a plurality of scans of the long bone. The mapping may include creating three-dimensional reconstructions from the plurality of scans of the long bone. The mapping may initially or additionally include an empirical and/or computer modeling step for obtaining one or more of: a length of the diaphysis of the long bone, medullary canal diameters of the long bone, diaphyseal lengths of the long bone, metaphyseal lengths of the long bone, and/or a cortical bone thickness of the long bone. These may be repeated for one or more bones of a same mammal, and/or for one or more mammals, in which the mammals are further identified (and/or categorized) by one or more of strain, sex, age, and biologic condition (e.g., a strain specifically altered to enhance, promote, remove, delay or otherwise effect one or more biologic conditions, and/or biologic components, such DNA, RNA, protein, fatty acid, enzyme, other biologic or chemical component, in which altering may include modifying expression, production, removal, and/or homeostasis, as compared with the unaltered strain). Said mapping provides the means for generating a reproducible length of the device for that long bone, based on at least a length of the diaphysis (in which the device may or will be sized for each age, sex and strain to be about or just slightly less, such as 5% to 30% less, than the length of the diaphysis), and/or based on the cortical thickness mid diaphysis (in which the device may or will be sized for each age, sex and strain to be about or less than about ⅓ the thickness of the cortical bone), and/or based on medullary canal diameters at regions of the medullary canal and/or as an average of one or more measurements (in which the device may or will be sized for each age, sex and strain to provide an interference fit when fitted in the medullary canal). Thus, in combination with the device 30 is a system for precisely sizing the device 30, which may be represented by an electronic mapping system (wired or wireless) that may include, for example, one or more of the following: desktop computer(s), laptop computer(s), tablet device(s), cellular telephone(s), one or more set top boxes, printer(s), and display(s). Fewer and/or additional components may also be included, such as servers, and or storage devices.

Generally, a mapping system will include a bus or other communication device to communicate information from one or more of elements of the system. For example, a processor is communicably coupled to a bus to process information. In addition, multiple processors and/or co-processors may be communicably coupled. The mapping system may further include random access memory (RAM) or other dynamic storage device (generally as a main memory), communicably operable with the bus. Random access memory may store information and instructions that may be executed by the one or more processors. Generally, the main memory may also be used to store temporary variables or other information (e.g., intermediate information) during execution of instructions by the one or more processors. In one embodiment, instructions are provided from the random access memory.

The mapping system may also include read only memory (ROM) and/or other static storage device operably coupled. In one embodiment, the read only memory is coupled via a bus. Such read only memory is for storing static information and instructions for the one or more processors. Each processor may also be associated with its own read only memory. A separate data storage device may also be coupled to the mapping system via a bus to store information and instructions. A data storage device includes, for example, a magnetic disk or optical disc and a corresponding drive. In some embodiments, instructions are provided to memory from a storage device (e.g., magnetic disk, a read-only memory, integrated circuit, CD-ROM, DVD) via a remote connection (e.g., over a network via a network interface) that is either a wired or wireless connection providing access to one or more electronically-accessible media. In some embodiments, hard-wired circuitry can be used in place of or in combination with software instructions. Execution of sequences of instructions is not limited to any specific combination of hardware circuitry and software instructions.

A computer-readable medium as described herein will include any mechanism or means of providing data (e.g., computer executable instructions) in a form readable by the communication device (e.g., a computer, a personal digital assistant, a cellular telephone). A computer-readable medium may include, as examples, but is not limited to: read only memory, random access memory, magnetic disk storage media, optical storage media, flash memory devices.

The mapping system may be further communicably coupled via a bus to a display device (e.g., cathode ray tube, liquid crystal display, light emitting diode, etc.). A display device displays certain data and information to an operator, user, or wearer. An input device with command selections with or without keys (e.g., key pad, key board, smart screen or pad, cursor control device, mouse, trackball, etc.) may be further coupled to the system via a bus, often to the one or more processors. For example, the input device communicates information and command selections to at least one processor. The input device may also control movement on a display (e.g., via a cursor). Navigation on a display may include screen buttons or links on a graphical user interface or keyboard buttons on a computer keyboard or by gesture inputs provided by a user (e.g., on or in association with an input pad).

The mapping system may be communicably coupled via a bus to one or more output devices (e.g., CT scanner, X-ray machine, MRI machine, etc.). Or the mapping system allows input of data obtained from such output devices. The output device will, for example, obtain scans or radiographs of the long bone (e.g. diaphysis of the long bone, as well as the long bone in cross-section at or about the mid diaphyseal region, and/or cross-section at or proximate the metaphyseal region). The scans or radiographs will be output to the mapping system, generally to the one or more processors. Said scans or radiographs and/or the data contained therein are analyzed, and/or used to generate reconstructed three-dimensional images, and/or stored in one or more memories or static devices, and/or used to generate one or more look-up tables comprising some or all of the data for the mapping system (e.g., categorizing and/or associating data with a mammal, and/or a bone type or name, and/or strain, sex, age, and/or biologic condition of the mammal). Additional algorithms allow precise optimization and production of one or more devices 30 based on bone type, age, gender, and strain of the mammal for which the device is to be fabricated for.

The mapping system may be used to prepare the one or more devices well in advance of their installation. For example, the mapping system may be used to prepare one or more kits containing one or a plurality of devices 30. For example, the kits may contain the one or more devices 30 precisely sized for a specific bone, a specific strain, a specific age and/or age range, for one or both genders, or any combination thereof. Each kit may further comprise any one or more of the surgical instruments to be used for installation of the one or more devices 30. The surgical instruments and accessories may include at least some or all of the following, many or most of which may be provided in sterile form: cloth drape (disposable), gloves, gauze, cotton swab, saline, isopropyl alcohol, clorhexidine/isopropyl alcohol, surgical disinfectant, surgical disinfectant applicator, eye ointment, razor, scalpel, periosteal elevator, calipers, forceps, Kern-style forceps, cutting wheel, hypodermic needle, tubing (e.g., 19 and/or 22 gauge), sutures, surgical adhesive, and a device as depicted in FIGS. 9A and 9B.

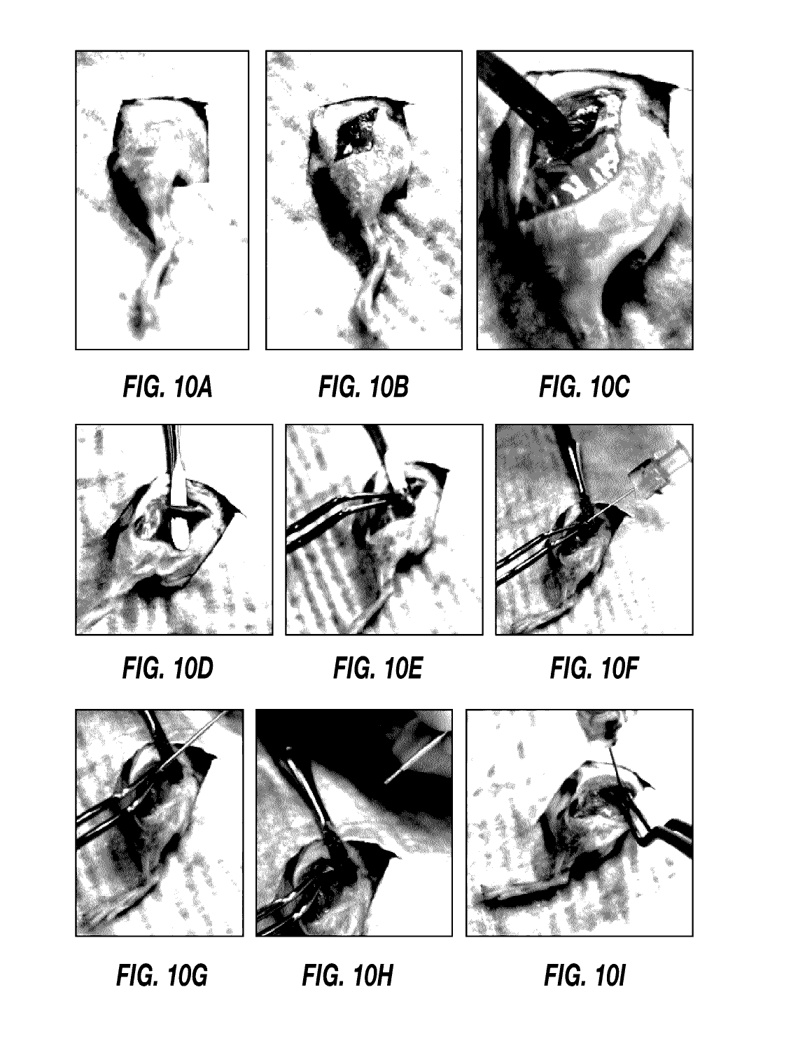

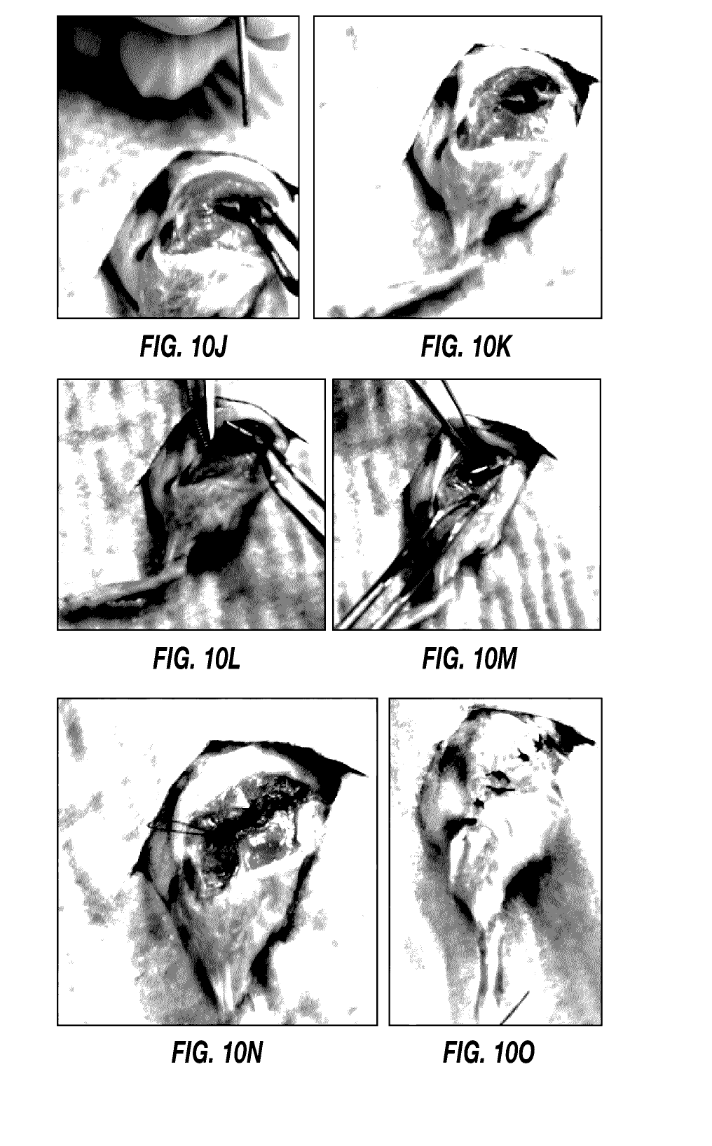

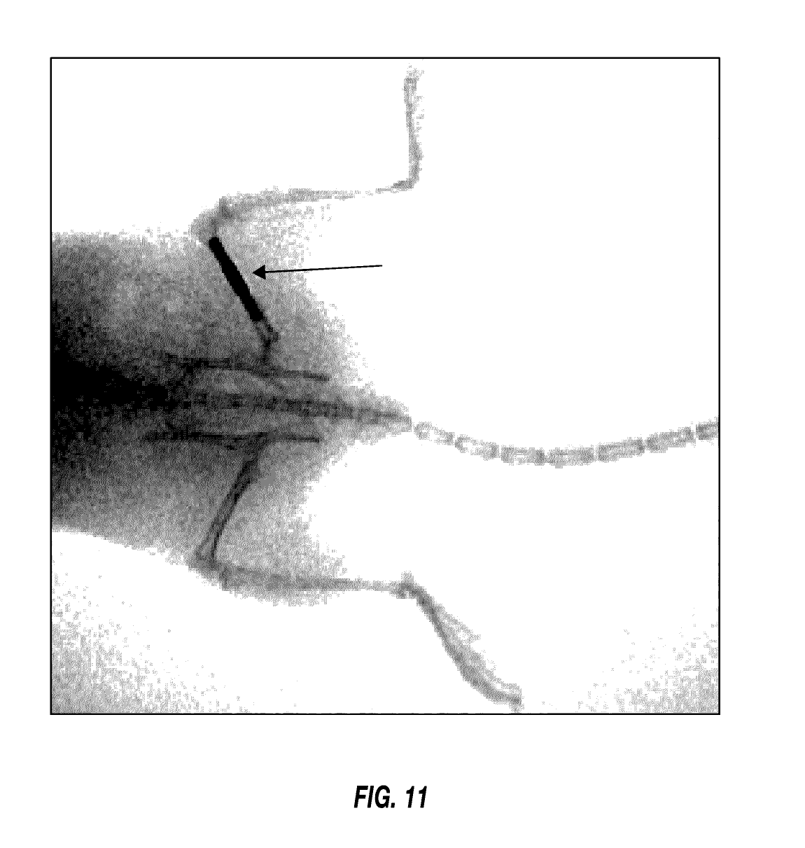

Device 30 installed in the medullary canal of the long bone of the mammal is depicted in FIGS. 10A to 10O. The installation procedures followed guidelines set by the Guide for the Care and Use of Laboratory Animals (8^{th} Edition), as well as additional policies set by local IACUC. Procedures are generally performed under sterile and/or sanitized conditions, such as in a surgical procedure room or clean laboratory with a closable door and no through-traffic, having a sterile field of approximately, which will depend on the size of the mammal. For example, for mice, the sterile field may be about 60×90 cm, as an example. When possible, the surface supporting the animal may be heated (e.g., with sanitized heating pad fitted to a warm water re-circulator on the drape and cover with sterile disposable drapes). Surgical equipment was arranged nearby. The surgical equipment for installation included a micro-drill, cutting wheel, absorbable suture, outer suture, scalpel blades, scalpel handle, wrapped sterile medullary devices 30, medullary depth gauge (e.g., using hypodermic tubing), fine nosed forceps, rat-teeth forceps, blunt reaming needles, needle drivers, small hemostats, fine scissors, periosteal elevator, modified Kern-style forceps. With the installation procedure described herein, it has been found that certain modifications to Kern forceps were beneficial, especially for installation of the device described herein in the long bone of small mammals, such as mice. The modification included having a distance between the forks of about 1 mm as depicted by the arrow in FIG. 9A. Another view of the Kern-style forceps modified as described herein is shown in FIG. 9B. Also, provided for installation of the device described herein were sterile cotton gauze (2×2 in), sterile Q-tips, sterile steel bowl (˜500 mL) containing sterile saline (0.9% w/v), and clorhexidine/isopropyl alcohol surgical disinfectant applicators.

Installation of the devices described herein was performed in mice. The process included anesthetizing the mammal (in accordance with veterinary guidance in the appropriate user manual). Sterile artificial tears lubricant ointment (e.g., 15% (v/v) mineral oil, 83% (v/v) white petrolatum) may be applied to the eyes. The mammal was adjusted so the hind-limb faced upwards, fur was removed (e.g., with an electric razor or hair removal cream), the site was wiped with sterile saline and a new fenestrated drape was placed covering all parts but the entire hind-limb (FIG. 10A). The proximal and distal ends of the long bone (e.g., femur as shown in the drawings) was identified and a 5-10 mm long incision was made in the longitudinal axis (FIG. 10B). The skin layer was separated from the fascia with a #15 scalpel, exposing a lateral approach to the long bone. For example, when the long bone is the femur, the biceps femoris and vastus lateralis will be exposed The septa where muscles meet was identified (it is a line of white tissue against the pink coloration of the muscle). With a scalpel, the intermuscular boundary was carefully disected until the long bone was visible. The incision was developed with a blunt periosteal elevator so as to expose the entire diaphysis (FIG. 10C). The elevator may be used to further expose the central two thirds of the long bone while taking care to preserve the posterior neurovascular bundle on the medial side (FIG. 10D). Soft tissue was gently scraped off of the bone with a scalpel, and the bone was dried with a sterile cotton or equivalent. The center of the femur was identified with calipers if necessary and marked with a sterile scalpel or marker, then the defect size was marked. For instance, in the mouse, having a 3 mm size defect, 1.5 mm was marked proximally and distally from the center. The long bone was grasped using a pair of fine-nosed forceps previously fashioned in the Kern style to prevent excessive pressure on the bone. These forceps may be initially tested in another specimen or mammal prior to use to ensure the bone will not break under pressure required for immobilization during cutting. A fine drill was fitted with a fine diamond-grit coated cutting wheel (e.g., 8 mm diameter×0.1 mm width), and the bone was cut, with a first cut while the elevator was place to protect tissue below (FIG. 10E). While raising the cut long bone (e.g., to about 45°) while firmly holding the extremity of the diaphysis, a second cut was made to remove the segment of the long bone. In the representative example, the defect size was 3 mm, hence a 3 mm segment of bone was removed, leaving two exposed segments of bone in the mammal, a proximal end and a distal end. The 3 mm defect size was found to meet the criteria found by others (e.g., see, Key, J. The effect of local calcium depot on osteogenesis and healing of fractures. J. Bone Joint Surg. (Am) 16, 176-184 (1934)). In some embodiments, the gap should not be less than about 1.5 mm for a small mammal long bone, such as the femur in a mouse at six weeks, as others have demonstrated that a gap as narrow as 1.8 mm does not sufficiently heal after 10 weeks and this could be delayed to 15 weeks with stripped perichondrium (see, e.g., Garcia, P. et al. Development of a reliable non-union model in mice. J Surg Res 147, 84-91, doi:10.1016/j.jss.2007.09.013 (2008)).

With the bone immobilized by forceps, the medullary cavity from the proximal end of the exposed bone and from the distal end of the other exposed bone were both carefully fitted and/or inserted and/or reamed with a suitably wide member (FIGS. 10F to 10K). For example, for the femur of mice, a blunt 23 G hypodermic needle may be used. The wide member may be one that is pre-made with a depth gauge (e.g., using tubing). Alternatively, a depth gauge may be used after fitting and/or inserting and/or reaming to ensure that the depth of the fitted and/or inserted and/or reamed medullary cavity is the length desired. This depth is generally to the end of the diaphyseal region, which is known in advance, using the method described above. For example, for the long bone of mice known to have a 9 mm diaphyseal region, the insert depth or fit depth or ream depth on each bone segment will be 3 mm when the defect is 3 mm. The depth guage for a small mammal, such as mice, may be made from a length of 22 G tubing placed in 19 G tubing. Carefully insert the medullary device 30 described herein into the proximal then into the distal medullary cavities to bring the long bone back to its original length (FIG. 10L to 10 M, in which a representative device 30 described herein is shown). The device 30 having the band that is the same size as the defect will, hence, establish a stable 3 mm gap (FIG. 10M). If needed, a small amount of manual stress may be applied to achieve a good interference fit of the cortical bone with the proximal region 32 and distal region 34 of the device 30 rod. The device 30 should fits snugly into the medullary cavities of both segments of the long bone. The lips of the band 20 of device 30 that are exposed to the cortical bone should be proximate to, or may be flush. Preferably, there should not be any visible gap between the lips of band 20 of the device 30 and the cortical bone. The medullary cavity may need additional fitting and/or inserting and/or reaming if there is the visible gap.

The muscle and peripheral tissue are repositioned over the device 30 and the exposed skin is closed (e.g., with a continuous absorbable 5-0 suture) (FIG. 10N). The skin is then closed (e.g., with a number of square knots, using nylon 5-0 sutures) (FIG. 10O). A surgical adhesive may be used to seal the closed incision. Hindlimb mobility should be observed within a short time after the mammal regains consciousness (e.g., 5 to 10 minutes). Daily postoperative monitoring was performed.

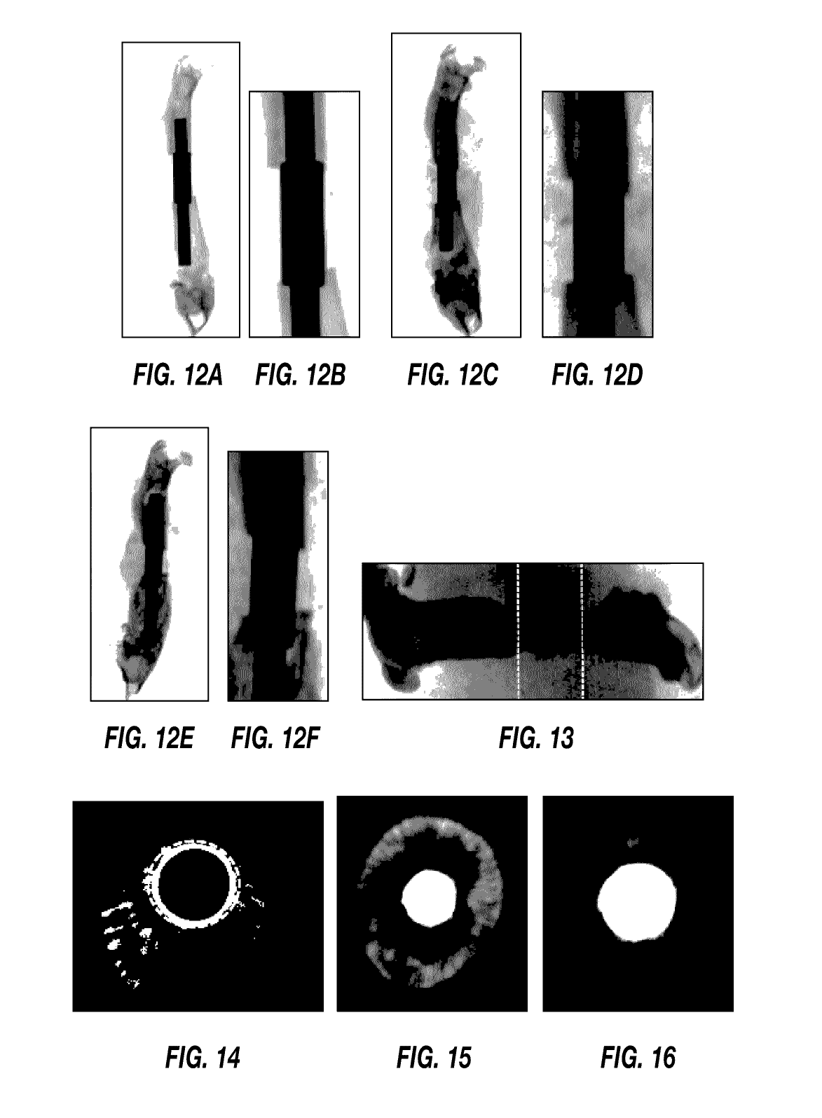

Live-mammal x-ray imaging may be performed during anesthesia to visualize pin placement and within 24 hours and thereafter after installation of the device described herein. FIG. 12 depicts proper placement of the device 30 described herein in a femur of a mouse. After about 5 days of post-operative monitoring, the small mammals may be returned to standard communal housing as per institutionally-approved policies. Sutures may be removed at about day 7 post-surgery.

Bone healing (e.g., repair and remodeling) was assessed using a specimen microCT (μCT) imager (e.g., Skyscan 1174). However, it is understood that a wide variety of methodologies may be used effectively. For radiographs depicted in FIGS. 12A-F, and 13 to 16, the microCT images was to the following parameters; voltage=29 kv, current=661 μA, power=19 watts, image pixel size (mm)=21.00; 360 degree rotation=yes; frame averaging=on (5); rotation Step (deg)=1.00, random movement=on. Images were stored as JPEG files and reconstruction software was used to generate axial images based on the following parameters; smoothing=on (4), misalignment compensation=on, ring artifact reduction=on (5), beam-hardening correction (40%), CS rotation (deg)=0.00. Set output to 2000-15,000 Hounsfield units.

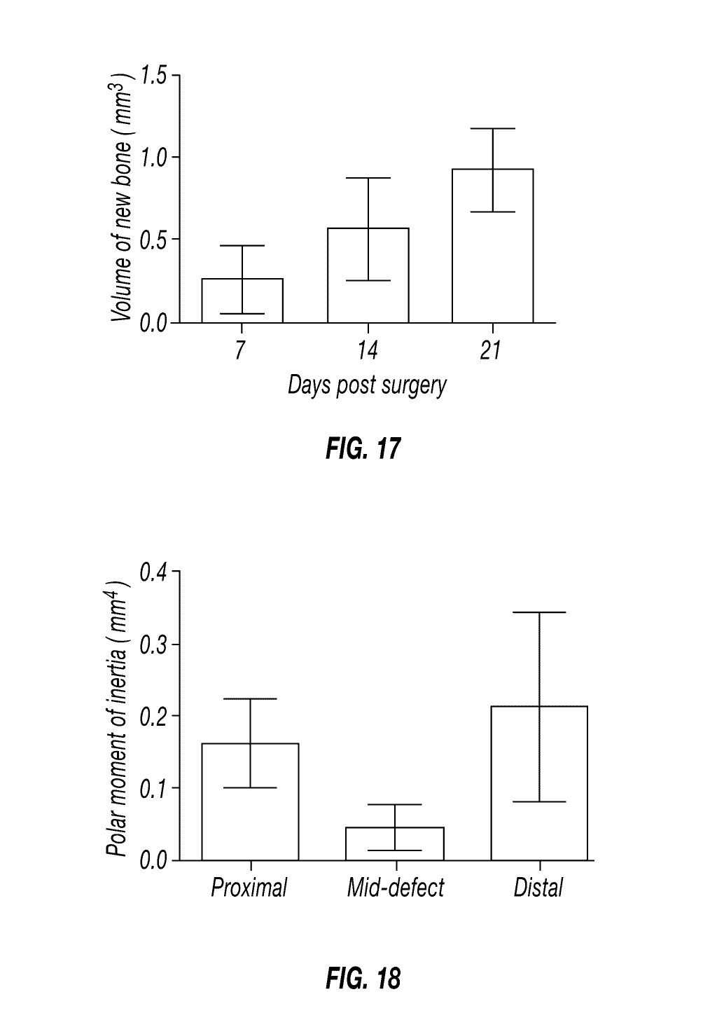

Using the axial images and analytical software, the region of interest (ROI) was defined by first setting the proximal and distal edges of the original defect. This was performed by selecting the sections that encompassed the band 20 only, as it is thicker and more are easily defined, as depicted in FIG. 13. Then the band 20 was excluded from calculations by drawing an exclusion zone around it (with a 100 μm margin), as depicted in FIG. 14, and transferring the zone to each of the sections in the ROI (FIGS. 15 and 16). Polar moments of inertia (FIG. 18), 3D reconstructions and calculations such as the volume of new bone (FIG. 17) were then performed.

Manual palpation of specimens confirmed that torsional and longitudinal motion was marginal after 7 days, while connective tissue accumulated around the device 30.

It was found that in the absence of additional therapeutic intervention, the edges of the defect typically extended about 0.5 mm during 21 days (FIGS. 12A to 12F) Bone growth arrested after this period as the inflammatory and anabolic stages of regeneration ceased resulting in a non-union defect. After 14-21 days of healing, the volume of de novo bone was readily determined from axial images generated by μCT scanning. (FIG. 17). Using the scanning, axial reconstruction and ROI selection procedures described above, the volume of new bone generated increased with time, but did not exceed 1 mm³ in the absence of additional therapeutic intervention, as compared with 6-7 mm³ new bone in an anatomically equivalent region of the uninjured long bone.

The polar moment of inertia (PMI), an estimation of the ability of a material to resist torsion based on cross-sectional area and density, was performed, as this has been shown to represent a suitable estimation of strength in long bones. Axial cross-sections at various distances from the lesion edges were selected for analysis. After 21 days, the PMI of de novo bone 0.25 mm from the lesion edges was in a range that was from between about 0.05 to 0.35 mm⁴, as compared with values of 0.02-0.08 mm⁴ at the center of the lesion for mice having the device 30 installed in the femur (FIG. 18). This confirmed the presence of non-union. The PMI of uninjured femur at an anatomically equivalent location typically ranges from between 0.5-0.7 mm⁴ under the conditions described here.

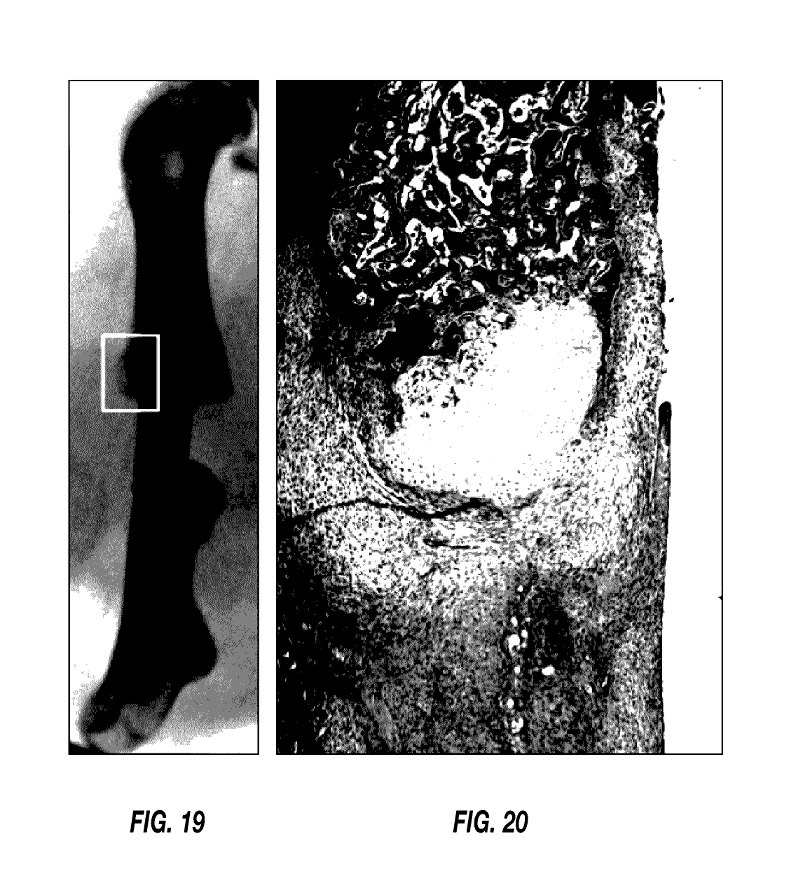

Decalcification of the tissues was monitored by x-ray scanning and also performed on specimens using Masson's trichrome-stained paraffin-embedded sections (FIG. 20) cut in the longitudinal direction as depicted in FIG. 19 following removal of the device 30 after the decalcification. The decalcification demonstrated bone outgrowth consisting of cartilage (ca) and cancellous bone (b) (scale bar: 0.5 mm). Further, the histological structure of the bone and connective tissue remained clear if the pin was removed carefully. FIG. 19 depicts methyl methacrylate embedding and sectioning of non-demineralized bone with the device 30 in place.

Described herein are improved intermedullary devices, and methods for configuring said device for bone, as well as methods for obtaining a stabilized defect with the described intermedullary device. In one or more embodiments, the devices described herein may suitably comprise, consist of, or consist essentially of an elongate member having a first end, a second end, and a mid region, the mid region comprising a band extending outwardly from the elongate member and increasing a cross-sectional diameter about at least a portion of the mid region, the band comprising an engaging surface configured for engaging with a cortical region of the long bone, the elongate member having a length from the first end to the second end that is about a length of the long bone's diaphyseal region. The device described herein may suitably comprise, consist of, or consist essentially of a device configured for positioning in a medullary canal of a long bone to divide the long bone into a proximal section having an exposed cortical region and a distal section having an exposed cortical region, with a spaced apart region separating the proximal section and the distal section, the device having a cross-sectional diameter at a mid region that is at least 10% greater than any other cross-sectional diameter of the device, and is so configured to engage with one or more of at least a portion of the exposed cortical region of the proximal section of the long bone and at least a portion of the exposed cortical region of the distal section of the long bone.

In one or more embodiments, the device may suitably comprise, consist of, or consist essentially of an elongate member having a first end, a second end, and a mid region. The mid region may comprise, consist of, or consist essentially of a band or collar extending outwardly away from an outer surface of the device to increase all or a portion of the cross-sectional diameter of the mid region of the device. The elongate member comprises, consists of, or consists essentially of a length that is about the same as, or is less than a length of a long bone's diaphyseal region for which the device is configured for. The band or collar of the device further comprises, consists of, or consists essentially of at least one engaging surface configured for engaging with a cortical region of the long bone at a defect site. Use of the device facilitates healing of the long bone at or near the defect site. A method of use of the device comprises, consists of, or consists essentially of configuring a first end of the device for fitting in a medullary canal at a first exposed region of the long bone, the first exposed region being at a defect site. Such a method further comprises, consists of, or consists essentially of configuring a second end of the device for fitting in a second exposed region of the medullary canal of the long bone, the second exposed region being at a defect site.

The methods may use standard laboratory and veterinary equipment. In one or more embodiments, the methods described herein may suitably comprise, consist of, or consist essentially of facilitating healing of a long bone using an intermedullary device, the method comprising, consisting of, or consisting essentially of, providing a first end of the intermedullary device in a first exposed region of a medullary canal of the long bone, the first exposed region formed at a mid portion of the long bone, the intermedullary device having a mid region with a cross-sectional diameter that is at least 10% greater than any other cross-sectional diameter of the intermedullary device; providing a second end of the intermedullary device in a second exposed region of the medullary canal of the long bone, the second exposed region formed at a mid portion of the long bone; causing cortical bone about the first exposed region to be proximate to a first engaging surface of the mid region of the intermedullary device; and causing cortical bone about the second exposed region to be proximate to a second engaging surface of the mid region of the intermedullary device.

The described intermedullary device is positioned or configured to position into the medullary canal without additional fixation, making the procedure technically more feasible and possible than other, alternative and more complicated approaches that employ external fixators and/or interlocking screws. Careful attention to the cross-sectional diameter of the proximal and distal regions of the device described herein minimizes torsional motion that occurs in other, alternative devices. Motion is particularly detrimental during the early stages of healing, which is prevented as described herein with the described device. The interference fit between the device described herein and endosteum (upon adequate fitting and/or reaming of the medullary canal), as described herein, along with other features of the device described herein (e.g., the larger cross-sectional diameter of the band and its precise length) minimizes torsional motion of the device. With selection of the appropriate device described herein and sizes based on strain, age and gender matching, the fit of the device described herein was found to be reproducibly robust, if not immediately, within a few days. Torsional motion of the fitted device may be further reduced by incorporating roughened surfaces and/or barbed attachment sites, as was described previously. With the systems and processes described, the device fabrication may be optimized for virtually any mammal, such as the more difficult mammals, including inbred mice, irrespective of natural or experimental bone phenotype.

The described intermedullary devices described herein prevented aberrant narrowing of the defect and damage of the bone extremities through longitudinal slippage. Said devices described herein also provide landmarks that define the original edges of the defect. As such, volumetric and PMI measurements may be made easier when imaging, such as when using CT scanning or other alternative imaging means. CT scanning, or alternatives means (e.g., evaluation by objective assessment of orthogonal x-ray images, 2D image analysis techniques, etc.) permit a level of quantitation that is not easily obtained with standard non-critical sized fracture techniques that often exhibit variable or poorly defined injuries. This, alone or in combination with histologic evaluation of specimens, alleviates sampling issues frequently faced with histomorphometric analyses of large mammal fractures. Furthermore, the permitted healing time (repair and remodeling) was relatively short, at 3 weeks for the mice, which is beneficial for large types of analyses,

In the claims, the word “comprising” does not exclude other elements or steps, and the indefinite article “a” or “an” does not exclude a plurality. The mere fact that certain measures are recited in mutually different dependent claims does not indicate that a combination of these measured cannot be used or used to an advantage.

While the invention has been illustrated and described in detail in the drawings and foregoing description, such illustration and description are considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

What is claimed is:

1. An intermedullary device configured for fixation of a long bone comprising:
an elongate member having a first end, a second end, and a mid region,
the mid region comprising a band extending outwardly from the elongate member, and increasing a cross-sectional diameter about at least a portion of the mid region,
the band comprising an engaging surface configured for engaging with a cortical region of the long bone,
the elongate member having a length from the first end to the second end that is about or near about a length of a diaphyseal region of the long bone.

2. The intermedullary device of claim 1, wherein the device in use is so configured that it does not penetrate one or more of a proximal epiphyseal region of the long bone or distal epiphyseal region of the long bone.

3. The intermedullary device of claim 1, wherein the device in use is so configured that it does not damage a metaphyseal region of the long bone.

4. The intermedullary device of claim 1, wherein the band has a length that is from about 15% to about 50% of a length of the intermedullary device.

5. The intermedullary device of claim 1, wherein the band comprises more than one band.

6. The intermedullary device of claim 1, wherein the intermedullary device is configured for use without an external fixation element.

7. The intermedullary device of claim 1, wherein the intermedullary device is configured for use without anchoring bores therein, or without a separate anchoring element.

8. The intermedullary device of claim 1, wherein the band is comprised of a same material as the elongate member.

9. The intermedullary device of claim 1, wherein the band is comprised of a different material than the elongate member.

10. The intermedullary device of claim 1, wherein the elongate member, excluding the band, has on average a cross-sectional diameter that is about or similar to an average cross-sectional diameter of a medullary canal of the long bone.

11. The intermedullary device of claim 1, wherein the intermedullary device further comprises one or more surface features, including one or more of the group comrpising a texturing, a roughening, a groove, a slit, a tab, and a barb.

12. The intermedullary device of claim 1, wherein the intermedullary device further comprises one or more surface coatings applied on at least a portion of the intermedullary device.

13. A device configured for positioning in a medullary canal of a bone to separate the bone into a proximal section having an exposed cortical region, a distal section having an exposed cortical region, and a spaced apart region separating the proximal section of the bone from the distal section of the bone, the device comprising:
a first end;
a second end; and
a mid region, in which a cross-sectional diameter of the mid region is at least 10% greater than any other cross-sectional diameter of the device,
the mid-region being so configured to reside in the spaced apart region separating the proximal section of the bone from the distal section of the bone, and to engage with at least a portion of the exposed cortical region of the proximal section of the bone, and to further engage with at least a portion of the exposed cortical region of the distal section of the bone, and
the device further comprising a length so that the first end extends to a distal end of the diaphyseal region and is proximate a distal metaphyseal region of the bone without penetrating the distal metaphyseal region, and the second end extends to a proximal end of the diaphyseal region and is proximate a proximal metaphyseal region of the bone without penetrating the proximal metaphyseal region.

14. The device of claim 13, wherein the device further comprises a first groove between the first end and the mid region, and a second groove between the second end and the mid region.

15. The device of claim 13, wherein the mid region of the device has a length that is from about 15% to about 50% the length of the device, and the mid portion spans a distance formed by the spaced apart region that separates the proximal section of the bone from the distal section of the bone.

16. A method of fixating a long bone with an intermedullary device, in which the long bone comprises a proximal portion having a first medullary canal and a first exposed region, and a distal portion having a second medullary canal and a second exposed region, the method comprising:
configuring a first end of the intermedullary device for a first medullary canal at the first exposed region, the first exposed region being at or near a mid portion of the long bone, the intermedullary device comprising a mid region and a cross-sectional diameter in the mid-region being at least 10% greater than any other cross-sectional diameter of the intermedullary device that provides a first engaging surface at a first end of the mid region of the intermedullary device and provides a second engaging surface at a second end of the mid region of the intermedullary device;
configuring a second end of the intermedullary device for a second medullary canal at the second exposed region;
causing at least a portion of the mid region of the intermedullary device to be configured to abut at least a portion of the first exposed region after having been configured for the first medullary canal of the proximal portion of the long bone in a manner for cortical bone at or near the first exposed region to be proximate to the first engaging surface of the first end of the mid region of the intermedullary device; and
causing at least a portion mid region of the intermedullary device to be configured to be proximate to at least a portion of the second exposed region after having been configured for the second medullary canal of the distal portion of the long bone in a manner for cortical bone at or near the second exposed region to be proximate to the second engaging surface of the second end of the mid region of the intermedullary device.

17. The method of claim 16, wherein configuring the first end of the intermedullary device for the first medullary canal at the first exposed region includes extending a length of the first end of the intermedullary device so the first end is at or is proximate to a diaphyseal region of the proximal portion of the long bone without penetrating the diaphyseal region of the proximal portion, and wherein configuring the second end of the intermedullary device for the second medullary canal at the second exposed region includes extending a length of the second end of the intermedullary device so the second end is at or is proximate to a diaphyseal region of the distal portion of the long bone without penetrating the diaphyseal region of the distal portion.

18. The method of claim 16, wherein causing at least a portion of the mid region of the intermedullary device to abut at least a portion of the first exposed region includes configuring the intermedullary device so at least a portion of the cortical bone abuts the first engaging surface of the first end of the mid region of the intermedullary device.

19. The method of claim 16, wherein causing at least a portion mid region of the intermedullary device to be proximate to at least a portion of the second exposed region includes configuring the intermedullary device so at least a portion of the cortical bone abuts the second engaging surface of the second end of the mid region of the intermedullary device.

20. The method of claim 16 further comprising locking the intermedullary device at the mid region without any external fixation elements and without any separate anchoring elements.
